(19) United States
(12) Patent Application Publication
(10) Pub. No.: US 2010/0075441 A1
(43) Pub. Date: Mar. 25, 2010

(54) RAPID IMMUNOCHROMATOGRAPHIC DETECTION BY AMPLIFICATION OF THE COLLOIDAL GOLD SIGNAL

(75) Inventors: Adnan Badwan, Amman (JO); Murshed Abdel-Qader Mohammed, Amman (JO)

Correspondence Address: PATENT CENTRAL LLC;Stephan A. Pendorf, 1401 Hollywood Boulevard, Hollywood, FL, 33020, US

(73) Assignees: The Jordanian Pharmaceutical Manufacturing Co., Naor (JO); Terramark Markencreation GmbH, Bremen (DE)

(21) Appl. No.: 12/518,748

(22) PCT Filed: Dec. 6, 2007
(86) PCT No.: PCT/EP2007/010620

§ 371 Date: Oct. 29, 2009

(30) Foreign Application Priority Data
Dec. 11, 2006 (EP) 06025530.4

(51) Int. Cl. G01N 33/553; C07K 17/14; G01N 33/558; G01N 33/543; G01N 33/58; C12Q 1/68; B23P 11/00

(52) U.S. Cl. 436/525; 530/391.1; 422/70; 422/61; 29/428

(57) ABSTRACT

The present invention relates to a method for rapid immunochromatographic detection of a target in a sample, wherein the target is an antibody and/or an antigen, using different colloidal gold conjugates conjugated with a specific antibody and some oligonucleotides and their complementary oligonucleotides and/or antibodies and their related antigens, rapid immunochromatographic detection devices, uses of the method for detecting diseases or specific conditions, and a method for the manufacture of the devices as well as a kit which comprises the devices.

The present invention relates to a method for rapid immunochromatographic detection. More precisely, the present invention relates to a method for rapid immunochromatographic detection of a target in a sample, wherein the target is an antibody and/or an antigen, using different colloidal gold conjugates for sensitivity enhancement by signal amplification. The present invention further refers to rapid immunochromatographic detection devices, to the uses of the method for detecting diseases or specific conditions, and to a method for the manufacture of the devices as well as to a kit which comprises the devices.

### BACKGROUND OF THE INVENTION

In recent years, the in vitro diagnostics (IVD) industry has made enormous efforts to develop immunochromatographic tests. Such tests have found applications in both clinical and non-clinical fields (1). A clinical utility of this test format has been shown for more than 150 different analytes, and many of them are targets now of commercially available diagnostic products (3). The wide range of applications for such devices has been reviewed (1, 2).

Rapid immunochromatographic test devices, e.g. in the form of a test strip, are made up of a number of components, see FIG. 1a. Such a test strip 101 commonly includes a sample pad 102, a conjugate pad 103, a membrane 104, e.g. a nitrocellulose membrane, and an absorbent pad 105. The membrane 104 is usually attached by means of an adhesive 106 to a supporting backing 107, e.g. made of plastic. In practice, the user dispenses a patient sample (usually urine or whole blood) onto the sample pad 102. The sample then flows through the sample pad 102 into the conjugate pad 103, where it mixes with and releases the detector reagent. This mixture then flows across the membrane 104, where it binds with the test and control reagents located in the capture test zone 108 (sample zone) and negative control zone 109, respectively. When the mixture binds to the reagent that forms the test line, a positive result is indicated. The colour intensity of the test line is proportional to the concentration of analyte in the sample. Excess sample that flows beyond the test and control zones 108, 109 is taken up in the absorbent pad 105.

Rapid immunochromatographic test devices for diagnostic purposes are easy to operate and thus do not only contribute to the comfort of professional users, e.g. medical stuff, but also allow the operation by non-professionals users, e.g. most patients.

However, despite the wide use of rapid immunochromatographic test devices, their suitability is still limited with regard to certain applications. Urine, for example, contains very low levels of IgG, frequently around 1 mg/l. Therefore, the detection of antibodies, e.g. directed to HIV or HCV, requires very sensitive techniques. To date, the tests for antibodies in urine samples are based on ELISA and Western blot techniques, which are labour-intensive, time-consuming and need to be carried out by qualified persons. Efforts are being made to develop simple and/or rapid tests for the detection of antibody to HIV in urine specimens (4).

Oral fluid specimens consist often of saliva, which predominantly contains IgA class antibody, and oral mucosal transudates, which mostly contain IgG, and therefore also have much lower levels of IgG than serum. The levels of IgG normally found in oral fluid specimens (approximately 15 mg/l) are, however, higher than in urine specimens and innovative simple and rapid technology that has been shown to be effective for whole blood, serum and plasma, e.g. lateral flow through a chromatographic membrane, has been developed for use with these specimens (4).

Human chorionic gonadotropin (hCG) is a glycopeptide hormone produced by the placenta during pregnancy. The appearance and rapid increase in the concentration of hCG in the subject's urine makes it a good marker for confirming pregnancy. The concentration of hCG in urine increases steadily to a circulation peak of as much as 50,000 mIU/ml between the eighth and eleventh weeks.

Urine hCG levels during pregnancy are estimated to be:

1. 10-30 mIU/ml 7-10 days post conception.
2. 37,000-50,000 mIU/ml 8-11 weeks after last menstrual period.
3. <5 mIU/ml Healthy men or non-pregnant women.

In the prior art the hCG test is a chromatographic immunoassay which uses specific antibodies to selectively identify hCG in urine with a high degree of sensitivity. Elevated levels of hCG as low as 20 mIU/ml can be detected within 3 minutes.

There are several tests used to detect the presence of hepatitis B antibodies. There are also several tests that detect the presence of viral antigens.

The hepatitis B surface antibody (anti-HBs) is the most common test. Its presence indicates previous exposure to HBV, but the virus is no longer present and the person cannot pass on the virus to others. The antibody also protects the body from future HBV infection. In addition to exposure to HBV, the antibodies can also be acquired from successful vaccination. This test is done to determine the need for vaccination (if anti-HBs is absent), or following the completion of vaccination against the disease, or following an active infection.

Hepatitis B surface antigen (HBsAg) is a protein antigen produced by HBV. This antigen is the earliest indicator of acute hepatitis B and frequently identifies infected people before symptoms appear. HBsAg disappears from the blood during the recovery period. In some people (particularly those infected as children or those with a weak immune system, such as those with AIDS), chronic infection with HBV may occur and HBsAg remains positive.

To test for human immunodeficiency virus (HIV) is an essential component in the diagnosis and treatment of persons infected with the virus, in screening of blood for transfusion, in surveillance and in HIV/AIDS related research. Thus accurate and cost-effective testing is of great importance in combating the spread of HIV. It is imperative that tests for the diagnosis of HIV infection be as accurate as possible, given the serious ethical, legal and social issues that accompany HIV infection.

The number of people living with HIV has now risen to reach its highest level ever: close to 40 million people are living with the virus and close to 5 million people were newly infected with HIV in 2004 alone. Worldwide, the AIDS epidemic killed over 3 million people last year alone (Source: UNAIDS). Furthermore, only one in five people needing HIV prevention worldwide have access to basic prevention services and only one in ten people living with HIV has been tested for the virus.

The HIV virus is most easily transmitted to others during the initial period of acute HIV infection, when the viral load (quantity of HIV RNA in the blood) is especially high and when people are not aware of being contaminated by the virus. Most HIV infections are transmitted at this stage, called primary infection. Earlier detection using ultra sensitive tests avoids missing primary infections, enabling immediate precautionary measures to be taken to help prevent the risk of HIV transmission to a non-infected partner, to an unborn child, or through blood donations or direct blood contact. Earlier detection of HIV infection also ensures the implementation of early antiretroviral therapy (ART) to slow down the progression of HIV infection, thereby improving patient care and quality of life.

The diagnosis of HIV infection is usually made on the basis of the detection of HIV antibodies and/or antigen. The diagnosis of an HIV infection can be made indirectly, i.e. through the demonstration of virus-specific antibodies. Besides such indirect diagnosis based on detection of antibodies, a direct diagnosis of HIV infection is also possible: either through the demonstration of infectious virus (using cell culture), viral antigens (p24 antigen ELISA) or viral nucleic acid (i.e. viral genome); the latter is also termed nucleic acid testing (NAT).

One important problem of HIV antibody testing is the so-called “diagnostic window”. This is the time period that elapses between the times of acquisition of HIV infection until detectable levels of antibodies are present. The switch from antibody-negative to antibody-positive is called “seroconversion”.

The most widely used screening tests are ELISAs as they are the most appropriate for screening large numbers of specimens on a daily basis, e.g. blood donations. The earliest assays used purified HIV lysates (1st generation assays). Improved assays based on recombinant proteins and/or synthetic peptides, which also enabled the production of combined HIV-1/HIV-2 assays, became rapidly available (2nd generation assays). The so-called 3rd generation or antigen-sandwich assays, which use labeled antigens as conjugate, are more sensitive and have reduced the diagnostic window period considerably (5, 6).

Tuberculosis (TB) is a major and increasing public health problem in both industrialized and developing countries. Hence, the development of new inexpensive, rapid and field adapted methods for its diagnosis is urgently needed. Sputum culture, which is still the reference method for the diagnosis of pulmonary TB, is cumbersome and time-consuming, and requires access to expensive biosafety level 3 (BSL3) laboratories. Microscopy of direct smears for acid-fast bacilli (AFB) as recommended by WHO for developing countries is the most commonly used method for diagnosis of TB. A major disadvantage with this method is its low sensitivity, even after concentration of the sputum samples.

The availability of new field adapted, low-cost, and rapid diagnostic tests to supplement AFB microscopy, and especially methods improving the diagnosis in AFB-negative disease, would be of great benefit for TB control programs, in particular in areas lacking appropriate safety laboratories. Among the newly developed methods for rapid diagnosis of TB, nucleic acid amplification methods such as PCR seem most promising, but the technology is still too complex to be feasible for TB control programs in developing countries. Antibodies against a number of mycobacterial antigens have been identified in patients using a variety of immunological techniques, but no antibody test has so far reached sufficient sensitivity and/or specificity for routine diagnostic purposes. Detection of circulating or secreted Mycobacterium tuberculosis antigens seems attractive and has been explored in a number of studies. However, no satisfactory commercial test for mycobacterial antigens in serum or sputum is currently available.

The idea of identifying mycobacterial antigens in urine of TB patients is attractive for several reasons: urine is more readily obtainable than serum samples and urinary specimens do not carry the risks inherent to needles and blood-based laboratory work. Furthermore, if the urine specimens are boiled before handling, there is no need for BSL3 facilities.

In 1920s, mycobacterial antigens were detected in the urine of TB patients, and the diagnostic potential of such antigens was subsequently discussed by other scientists. More recently, the diagnostic value of mycobacterial antigens in the urine of leprosy patients has been assessed. Unfortunately, the techniques involved turned out to be insufficiently sensitive in paucibacillary patients, the patient group where improved diagnostic tests are most needed.

Lipoarabinomannan (LAM) is a major and structurally important glycolipid component of the outer cell wall of all mycobacteria and may account for up to 15% of the total bacterial weight. LAM is a carbohydrate antigen with glycosidic linkages for which no human degrading glycosidases are known. Hence, we assumed that in active mycobacterial disease LAM may be cleared through the kidneys and occur in urine in antigenically intact form. Furthermore, since LAM is a carbohydrate antigen and thus inherently heat-stable, LAM may be detectable by sensitive immunological techniques, even after boiling of the urine. At least theoretically, the amount of LAM in the urine should reflect the bacterial load, metabolic activity and/or rate of degradation of the bacteria, and hence permit a semi-quantitative assessment of the infectious status. A high sensitive, simple, fast and method for LAM detection and quantification was reported using an enzyme-linked immunosorbent assay (ELISA) in AFB positive sputa from TB patients (7).

It is an object of the present invention to overcome the problems with regard to the applicability of rapid immunochromatographic test devices for the detection of hCG, anti-lipoarabinomannan (LAM), HBsAG, anti-HBs, IgG, e.g. HIV antibodies, in urine, blood, serum or saliva by enhanced sensitivity.

### SUMMARY OF THE PRESENT INVENTION

It is an object of the present invention to solve the rapid immunochromatographic detection of a target in a sample by a new method especially through sensitivity enhancement and to overcome the disadvantages of the prior art.

In one embodiment the present invention concerns a method for rapid immunochromatographic detection of a target in a sample comprising the step of forming a sandwich by contacting
(a) a first colloidal gold conjugate conjugated with a specific antibody or antigen and at least one oligonucleotide or non-specific antibody or antigen, and
(b) a second colloidal gold conjugate conjugated with at least one complementary oligonucleotide or non-specific antibody or antigen.

Surprisingly, the object is achieved by the inventive method through signal amplification using different colloidal gold conjugates conjugated with a specific antibody and some oligonucleotides and their complementary oligonucleotides and/or antibodies and their related antigens. The rapid immunochromatographic detection method is using the oligonucleotides to multiply the colloidal gold signal by branched links between both colloidal gold conjugates.

In one embodiment the present invention further relates to a method, comprising the following steps of
(a) applying the sample to a sample application site,
(b) allowing the target in the sample getting captured by the first colloidal gold conjugate from a target site at a first conjugate releasing site,
(c) allowing the target in the sample to move to a test zone getting captured by another immobilized specific antibody or antigen from another target site,
(d) allowing to release the second colloidal gold conjugate from a second conjugate releasing site for capturing the first colloidal gold conjugate from at least one oligonucleotide site or non-specific antibody or antigen site,
(e) allowing the sample to move through the test zone and a control zone to an absorbent site,
(f) allowing to continuously release the first and the second colloidal gold conjugates from the first and the second conjugate releasing sites to propagate to the test zone and the control zone,
(g) detecting a color in the control zone, and
(h) detecting a color in the test zone.

In a further embodiment the present invention concerns a test device for conducting the method for rapid immunochromatographic detection of a target in a sample according to the present invention comprising a housing comprising a test strip comprising a sample application site; a first conjugate releasing site; a second conjugate releasing site; a nitrocellulose membrane; a test zone and a control zone; and a sample absorbent site, see FIG. 1b. The second conjugate releasing site may also be laminated within the upper side of the housing.

In another embodiment the present invention concerns another test device for conducting the method for rapid immunochromatographic detection of a target in a sample comprising a detection cup, see FIG. 4 and FIG. 5, comprising
(a) a sample collecting container
(b) a test strip comprising a sample application site; a nitrocellulose membrane; two test zones and a control zone; a sample absorbent site; and
(c) a first conjugate releasing site and a second conjugate releasing site separated from the test strip.

In another embodiment the present invention relates to the use of the method for diagnosing and monitoring a disease or a specific condition of a subject by detecting a target in a sample.

In a further embodiment the present invention refers to a kit for rapid immunochromatographic detection of a target in a sample comprising the test devices according to the present invention, reagents, wash buffers and a manual.

In a further embodiment the present invention concerns a method for the manufacture of the test device according to the present invention comprising the following steps of
(a) preparing a first colloidal gold conjugate by adding a specific antibody or antigen to a conjugation buffer with at least one oligonucleotide or non-specific antibody or antigen and then adding it to a colloidal gold solution,
(b) preparing a second colloidal gold conjugate by adding at least one complementary oligonucleotide or non-specific antibody or antigen in a conjugation buffer to a colloidal gold solution, and
(c) preparing a first conjugate releasing site and a second conjugate releasing site by applying the first and the second gold conjugate on different pads.

In another embodiment the present invention relates to a method for the manufacture of the test device according to the present invention comprising the following steps of
(a) preparing a sample application site, a test zone, a control zone and a sample absorbent site,
(b) assembling the sample application site, the test zone, the control zone, the sample absorbent site together with the first and the second conjugate releasing sites on a test strip,
(c) applying the first and the second gold conjugates on different sites of the same card separated by a divider, and
(d) assembling the test strip in a housing.

In a further embodiment the present invention relates to a method for the manufacture of the test device according to the present invention comprising the following steps of
(a) preparing a sample application site, a test zone, a control zone and a sample absorbent site,
(b) assembling the sample application site, the test zone, the control zone, the sample absorbent site together with the first conjugate releasing site on a test strip, and
(c) assembling the test strip and the second conjugate releasing site in a housing.

In another embodiment the present invention relates to a method for the manufacture of the test device according to the present invention comprising the following steps of
(a) preparing a sample application site, two test zones, a control zone and a sample absorbent site,
(b) assembling the sample application site, the test zones, the control zone, and the sample absorbent site on a test strip, and
(c) assembling the test strip and the first and second conjugate releasing sites separately in a detection cup.

The rapid immunochromatographic detection system comprises a detection test strip of two gold conjugate releasing sites or pads with different compositions. The first conjugate releasing site or pad 103.1 is laminated on the test strip between the sample application site or sample pad and the nitrocellulose membrane, while the second conjugate releasing site or pad 103.2 is laminated above the first conjugate releasing site separated by a divider 110 to be released directly toward the nitrocellulose membrane without flow through the first conjugate releasing site to avoid interaction with the first conjugate before reaching the membrane, see FIG. 1b. The second conjugate releasing site can also be laminated within the upper side of the plastic housing of the device. The first conjugate releasing pad contains a gold that is conjugated with the detection specific antibody/antigen and (at the same time) with some specific oligonucleotides, while the second conjugate releasing pad contains a gold conjugated with the complementary oligonucleotides that are conjugated with the first colloidal gold conjugate, see FIG. 2. Specific antibodies/antigens which should differ from the detection specific antibodies/antigens with their specific antigens/antibodies may be employed to play the same role as the oligonucleotides and their complementary oligonucleotides. The first conjugate releasing pad is containing an extra amount of Tween 20 that enables the first conjugate to be released before the second conjugate.

By the sample flow within the rapid immunochromatographic test the antibody on the first pad which contains the oligonucleotides will capture the antigen/antibody in the sample and carry it to be captured by the other antibody/antigen that is immobilized on the nitrocellulose membrane to form the sandwich detection. Then, the second conjugate releasing pad will release its gold that is conjugated with the complementary oligonucleotides. The last mentioned conjugate would bind with the first conjugate from the oligonucleotide(s) side. This binding could be happened by any of the conjugated oligonucleotides with its complementary oligonucleotide on the other gold conjugate. At the same time, the other oligonucleotides will be able to link with their complementary oligonucleotides besides the probability of capturing the first conjugate that will capture the second conjugate to form more and more branched bonds which propagates the accumulation of colloidal gold particles onto the capturing/sample line. This propagation and accumulation of colloidal gold signal will amplify the signal and highly increase the sensitivity. This will enable us to detect very low concentrations that are not detectable using the same technique without signal amplification.

### DETAILED DESCRIPTION OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purposes of the present invention, all references as cited herein are incorporated by reference in their entireties.

Preferably, the terms used herein are defined as described in “A multilingual glossary of biotechnological terms: (IUPAC Recommendations)”, Leuenberger, H. G. W., Nagel, B. and Kölbl. H. eds. (1996), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step.

As outlined above there is a need in the prior art to provide a new method for rapid immunochromatographic detection of a target in a sample for the detection of a disease or a specific condition such as pregnancy in a subject. There is also a need in the art for methods suitable for rapid and sensitive detection of an antibody and/or antigen having a higher sensitivity than methods from the prior art.

In a first aspect the present invention provides a method for rapid immunochromatographic detection of a target in a sample comprising the step of forming a sandwich by contacting
(a) a first colloidal gold conjugate conjugated with a specific antibody or antigen and at least one oligonucleotide or non-specific antibody or antigen, and
(b) a second colloidal gold conjugate conjugated with at least one complementary oligonucleotide or non-specific antibody or antigen.

The first colloidal gold conjugated with a first antibody or antigen captures the target in the sample and forms a complex “target-first colloidal conjugate”. Preferably this target in the sample is an antigen and/or antibody.

In a preferred embodiment of the method according to the present invention each gold conjugate comprises between 1 and 15 different oligonucleotides, e.g. 1, 2, 3, 4, 5, 6, 7, 8, 9, 10, 11, 12, 13, 14, 15, preferably between 2 and 10 different oligonucleotides, more preferably between 3 and 6 different oligonucleotides. In addition, the oligonucleotides conjugated with the second gold conjugate are complementary to the oligonucleotides of the first colloidal gold conjugate.

Mostly 2-4 oligonucleotides per gold conjugate are used. These oligonucleotides are of about 20-nucleotides in length. These oligonucleotides have an amino group at the 5′ terminus which is conjugated with bovine serum albumin. The bond between the gold and the oligonucleotides is the same as the one between the gold and the antibodies or antigens.

In a further first aspect the present invention provides a method comprising the following steps of
(a) applying the sample to a sample application site,
(b) allowing the target in the sample getting captured by the first colloidal gold conjugate from a target site at a first conjugate releasing site,
(c) allowing the target in the sample to move to a test zone getting captured by another immobilized specific antibody or antigen from another target site,
(d) allowing to release the second colloidal gold conjugate from a second conjugate releasing site for capturing the first colloidal gold conjugate from at least one oligonucleotide site or non-specific antibody or antigen site,
(e) allowing the sample to move through the test zone and a control zone to an absorbent site,
(f) allowing to continuously release the first and the second colloidal gold conjugates from the first and the second conjugate releasing sites to propagate to the test zone and the control zone,
(g) detecting a color in the control zone, and
(h) detecting a color in the test zone.

In one embodiment the method comprises further a first specific antibody or antigen which is selected from the group consisting of anti-beta chorionic gonadotropin hormone (anti-βhCG), anti-lipoarabinomannan (LAM), hepatitis virus antibodies against or antigens from hepatitis virus type A, hepatitis virus type B, or hepatitis virus type C or human immunoglobulin G antibodies or antigens.

Other antibodies and antigens which can be used are HIV specific antibodies or antigens, tuberculosis specific antibodies or antigens, malaria specific antibodies, toxoplasmosis specific antibodies or antigens, rubella specific antibodies, Leishmania specific antibodies or Pneumonia specific antibodies. Monoclonal antibodies are preferred, whereas polyclonal antibodies are applicable.

In one preferred embodiment the hepatitis virus antigen is hepatitis B surface antigen (HBsAg) and the hepatitis virus antibody is anti-HBsAg.

In one embodiment the method comprises further another specific antibody or antigen which is selected from the group consisting of anti-alpha chorionic gonadotropin hormone (anti-ahCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-O or HIV-1-M.

In one preferred embodiment the hepatitis virus antigen is hepatitis B surface antigen (HBsAg), the hepatitis virus antibody is anti-HBsAg and the human immunodeficiency virus (HIV) antigen is HIV p160.

Moreover, antigen detection antibodies are often pairs of monoclonal antibodies for example of anti-hepatitis B surface antigen (anti-HBsAg), anti-HIV p160 or anti-HIV p24.

In one embodiment of the method the sample comprises a body fluid of a subject.

In one preferred embodiment the body fluid is selected from the group consisting of urine, whole blood, serum, plasma and saliva.

In another aspect the present invention concerns a test device for conducting the method for rapid immunochromatographic detection of a target in a sample according to the present invention comprising a housing comprising a test strip 101 comprising a sample application site 102; a first conjugate releasing site 103.1; a second conjugate releasing site 103.2; a nitrocellulose membrane 104; a test zone 108 and a control zone 109; and a sample absorbent site 105.

The first conjugate releasing site or pad 103.1 is laminated on the test strip between the sample pad and the nitrocellulose membrane, while the second conjugate releasing site or pad 103.2 is located above the first conjugate releasing pad separated by a divider 110, in order to be released directly toward the nitrocellulose membrane without flow through the first conjugate pad to avoid interact with the first conjugate before reaching the membrane, see FIG. 1b. The second conjugate releasing site can be laminated within the upper side of the plastic housing of the device.

In one embodiment the test device further comprises the test strip 101 which is attached to a supporting backing 107 by means of an adhesive 106.

In a preferred embodiment the supporting backing 107 of the test device is a plastic backing.

In another embodiment the test zone 108 of the test device comprises another specific antibody or antigen.

In another preferred embodiment the test zone 108 of the test device comprises another specific antibody or antigen which is selected from the group consisting of anti-alpha chorionic gonadotropin hormone (anti-ahCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-O or HIV-1-M.

In a more preferred embodiment the hepatitis virus antigen is hepatitis B surface antigen (HBsAg), the hepatitis virus antibody is anti-HBsAg and the human immunodeficiency virus (HIV) antigen is HIV p160.

Moreover, antigen detection antibodies are often pairs of monoclonal antibodies for example of anti-hepatitis B surface antigen (anti-HBsAg), anti-HIV p160 or anti-HIV p24.

In another embodiment of the present invention the second conjugate releasing site 103.2 is laminated within the upper side of the housing.

In a further aspect the present invention concerns a test device for conducting the method for rapid immunochromatographic detection of a target in a sample according to the present invention comprising a detection cup 510.

In one embodiment the detection cup 510 of the test device comprises
(i) a sample collecting container 501
(j) a test strip comprising a sample application site 404; a nitrocellulose membrane 401; two test zones 403 and a control zone 402; and a sample absorbent site 405; and
(k) a first conjugate releasing site 503 and a second conjugate releasing site 504 separated from the test strip.

In one embodiment the test zones of the detection cup further comprise another specific antibody or antigen.

In a preferred embodiment another specific antibody or antigen is selected from the group consisting of anti-alpha chorionic gonadotropin hormone (anti-ahCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-O or HIV-1-M.

In one preferred embodiment the hepatitis virus antigen is hepatitis B surface antigen (HBsAg), the hepatitis virus antibody is anti-HBsAg and the human immunodeficiency virus (HIV) antigen is HIV p160.

Moreover, antigen detection antibodies are often pairs of monoclonal antibodies for example of anti-hepatitis B surface antigen (anti-HBsAg), anti-HIV p160 or anti-HIV p24.

In another aspect the invention relates to the use of the method for diagnosing and monitoring a disease or a specific condition of a subject by detecting a target in a sample.

In one embodiment the specific condition is pregnancy.

In a preferred embodiment the target of the specific condition is human chorionic gonadotropin hormone (hCG).

In another embodiment the disease is hepatitis selected of the group consisting of hepatitis type A, hepatitis type B, or hepatitis type C.

In a preferred embodiment the selected hepatitis type is hepatitis type B.

In a more preferred embodiment the target of the disease which is hepatitis type is hepatitis B surface antigen (HBsAg).

In another embodiment the disease is an HIV infection selected from the HIV infection group consisting of HIV type HIV-1 and HIV-2 or HIV subtype HIV-1-N, HIV-1-O or HIV-1-M.

In a more preferred embodiment the target of the HIV infection is selected from an HIV antibody or antigen selected from the group consisting of p41, p120, p160, p18, p24/25, p55, p34, p40, p52, p68.

In a further more preferred embodiment the HIV antigen is p160.

In a further aspect the invention concerns a kit for rapid immunochromatographic detection of a target in a sample comprising the test device comprising the housing according to the invention.

In one embodiment the kit comprises further reagents, wash buffers and a manual.

In another aspect the invention relates to a method for the manufacture of the test device according to the invention.

In one embodiment the method comprises the following steps of
(a) preparing a first colloidal gold conjugate by adding a specific antibody or antigen to a conjugation buffer with at least one oligonucleotide or non-specific antibody or antigen and then adding it to a colloidal gold solution,
(b) preparing a second colloidal gold conjugate by adding at least one complementary oligonucleotide or non-specific antibody or antigen in a conjugation buffer with to a colloidal gold solution, and
(c) preparing a first conjugate releasing site and a second conjugate releasing site by applying the first and the second gold conjugate on different pads.

In one embodiment the method further comprises the following steps of
(a) preparing a sample application site 102, a test zone 108, a control zone 109 and a sample absorbent site 105,
(b) assembling the sample application site 102, the test zone 108, the control zone 109, the sample absorbent site 105 together with the first 103.1 and the second conjugate releasing sites on a test strip 101,
(c) applying the first and the second gold conjugates on different sites of the same card separated by a divider 110, and
(d) assembling the test strip 101 in a housing.

In anther embodiment the method further comprises the following steps of
(a) preparing a sample application site 102, a test zone 108, a control zone 109 and a sample absorbent site 105,
(b) assembling the sample application site 102, the test zone 108, the control zone 109, the sample absorbent site 105 together with the first conjugate releasing site 103.1 on a test strip 101, and
(c) assembling the test strip 101 and the second conjugate releasing site 103.2 in a housing.

In a further embodiment the method comprises the following steps of
(a) preparing a sample application site 404, two test zones 403, a control zone 402 and a sample absorbent site 405,
(b) assembling the sample application site 404, the test zones 403, the control zone 402, and the sample absorbent site 405 on a test strip, and
(c) assembling the test strip and the first 503 and second conjugate 504 releasing sites separately in a detection cup 510.

### BRIEF DESCRIPTION OF THE FIGURES

FIG. 1a shows top and side views of a typical rapid-flow immunochromatographic test device in the form of a test strip 101 including a sample pad 102, a conjugate pad 103, a membrane 104, an absorbent pad 105, an adhesive 106, a supporting backing 107, a test zone 108, and a control zone 109.

FIG. 1b shows top and side views of our modified rapid-flow immunochromatographic test device in the form of a test strip 101 including a sample pad 102, a first conjugate pad 103.1, a second conjugate pad 103.2, a membrane 104, an absorbent pad 105, an adhesive 106, a supporting backing 107, a test zone 108, a control zone 109, and the two conjugates divider 110.

FIG. 2 shows the schematically view of the first and second colloidal gold, whereas the first colloidal gold 101 is conjugated with an antibody 202 and four different oligonucleotides 203, 204, 205, 206 and the second colloidal gold 211 is conjugated with four oligonucleotides 203′, 204′, 205′, 206′ complementary to the oligonucleotides of the first colloidal gold 101.

FIG. 3 shows the main principle of signal amplification:

By the sample flow within the rapid immunochromatographic test the target in the sample 220 will be captured by the antibody or antigen 202 of the first colloidal gold 201 to form the complex “target-first colloidal gold”. This complex flows to the test zone, where it will be captured by another antibody or antigen 108 that is immobilized onto the membrane 104 of the test zone to form a sandwich detection. Then, the second colloidal gold 211 conjugated with the complementary oligonucleotides 203′, 204′, 205′, 206′ to the oligonucleotides 203, 204, 205, 206 of the first colloidal gold will be released and will bind to the first conjugate from the oligonucleotide(s) side and enhance the signal.

FIG. 4 shows an assembly comprising a nitrocellulose membrane 401 with a control line 402 and a sample line 403, as well as a sample pad 404 and an absorbent pad 405.

FIG. 5 shows an immunochromatographic detection cup 510 comprising a sample collecting container 501, a cap 502 that contains the absorbent pad 405 and a similar assembly as shown in FIG. 4, except that there are two sample lines 403. Furthermore, there are the first conjugate releasing pad 503 and the second conjugate releasing pad 504 which are separately fixed from each other to the wall of the container.

### EXAMPLES

The following examples illustrate the present invention without, however, limiting the same thereto.

### Example 1

### Preparation of Oligonucleotide and Complementary Oligonucleotide Labeled Bovine Serum Albumin

5 mg of bovine serum albumin (BSA) was linked to each oligonucleotide (about 20 nucleotide having an amino group at 5′ terminus) and another 5 mg to complementary oligonucleotide (about 20 nucleotide having an amino group at 5′ terminus), according to a procedure comprising the following steps, according to the method described by Duncan et al. 1983 (7):

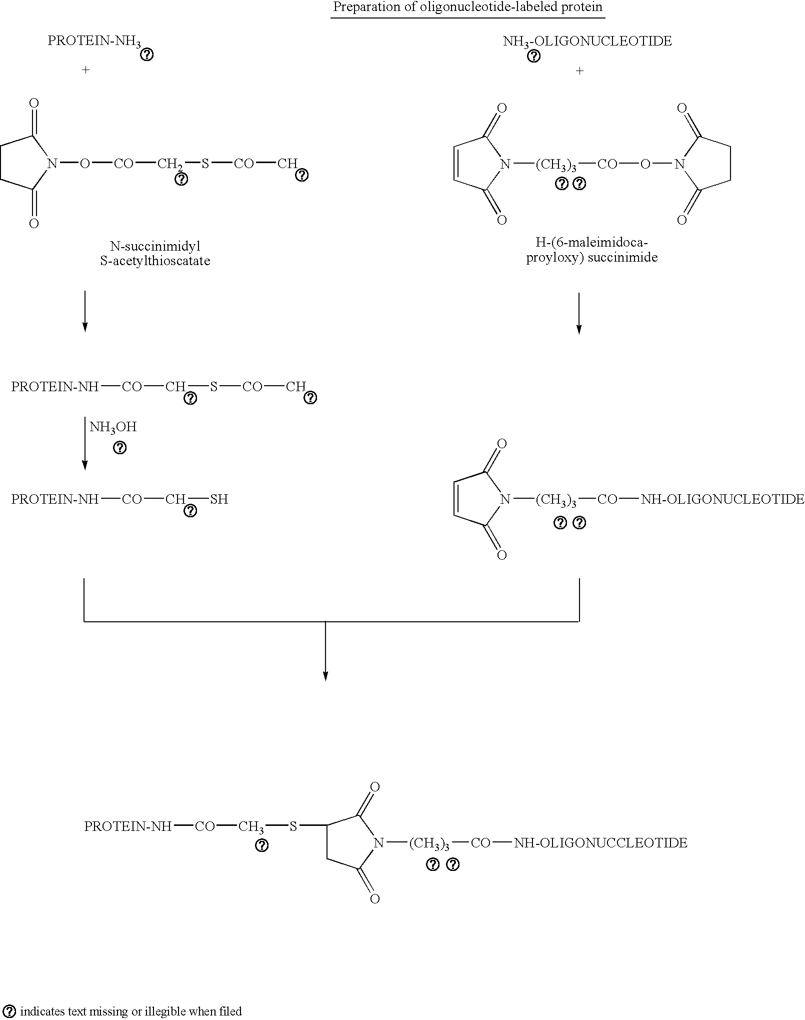

### Example 2

### Manufacturing Procedure of a Test Device

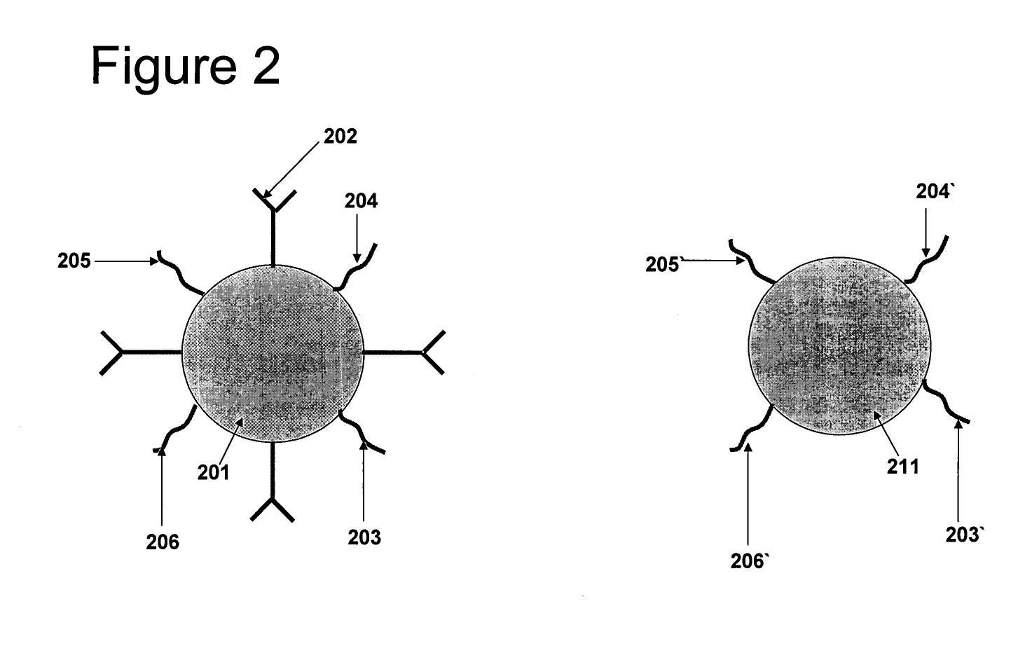

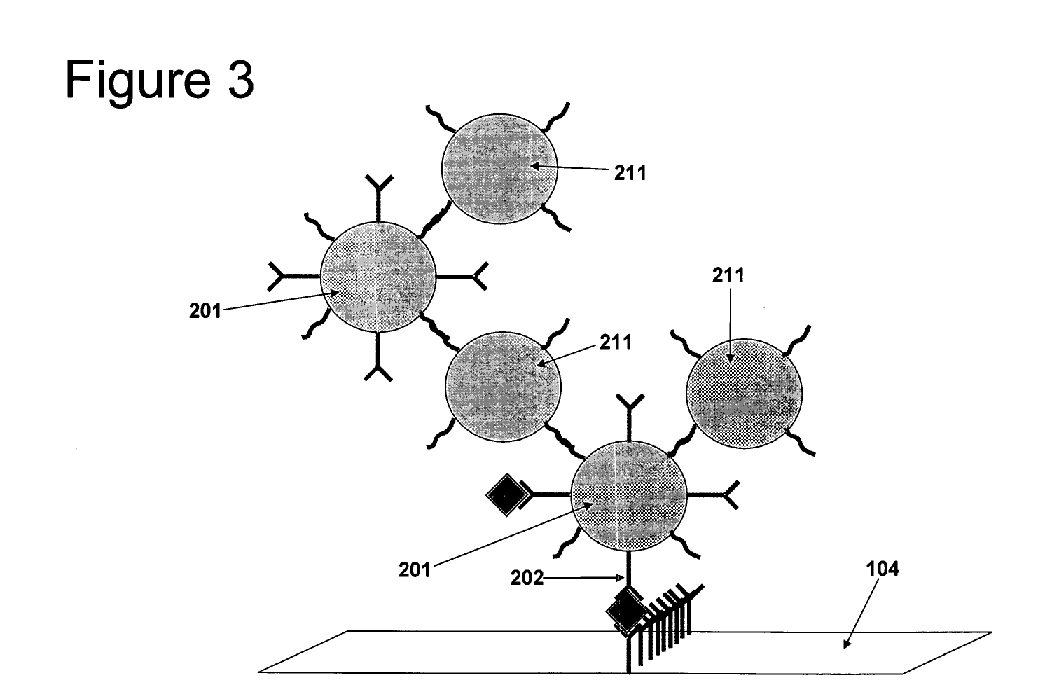

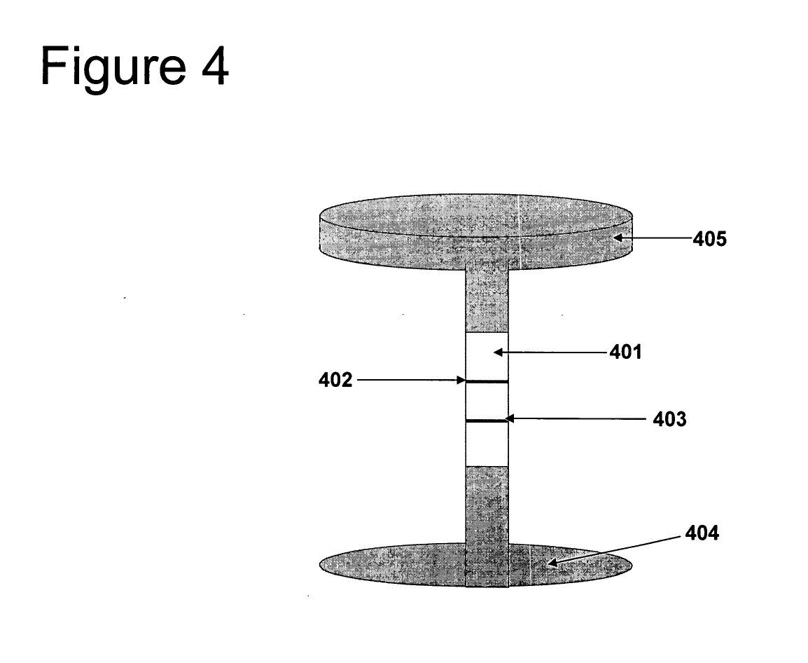

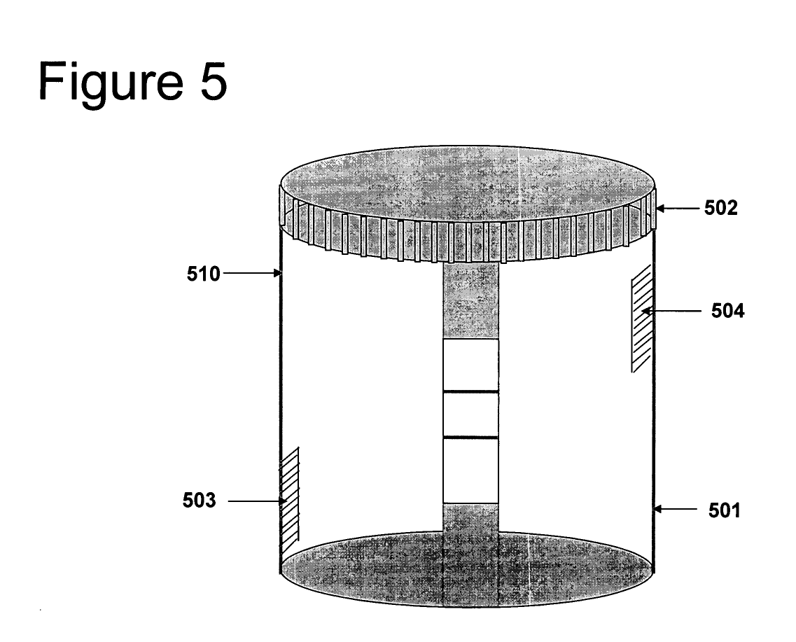

The oligonucleotide and complementary oligonucleotide labeled bovine serum albumin (BSA) prepared as described in Example 2 are further processed according to a procedure comprising the following steps:
1. Preparation of oligonucleotides* 203, 204, 205, 206-labeled bovine serum albumin (BSA) (solution 1), see FIG. 2, (solution 1).
2. Preparation of complementary oligonucleotides* 203′, 204′, 205′, 206′-labeled bovine serum albumin (solution 2), see FIG. 2, (solution 2).
3. Preparation of a 1% aqueous solution of tetrachloroauric acid at room temperature;
4. Preparation of a 4% trisodium citrate aqueous solution at room temperature;
5. Preparation of a 0.05 M potassium carbonate aqueous solution at room temperature;
6. Preparation of 400 ml of phosphate stabilizing buffer of pH 7.4 that contains BSA, Tween 20, Sucrose, polyvinylpurrolidone and preservative (like sodium azide) at room temperature;
7. Preparation of a colloidal gold solution by reduction of 1.7 ml boiling tetrachloroauric acid solution (after dilution into 100 ml) using 1 ml trisodium citrate solution and let it takes the room temperature;
8. Dilution of the colloidal gold solution as 1:1 using distilled water. Adjust the pH to 7.4 using potassium carbonate solution at room temperature;
9. Preparation of 200 ml of phosphate conjugation buffer of pH 7.4 at room temperature;
10. Partition of the 200 ml conjugation buffer by dividing it into two flasks (100 ml of each);
11. Addition of 0.5 mg of aqueous antibody (e.g. anti-hIgG or anti-βhCG) to the conjugation buffer in the first flask with stirring at room temperature;
12. Addition of 0.5 mg of oligonucleotides labelled BSA aqueous solution (solution 1) to the first flask at room temperature;
13. Addition of 1.0 mg of complementary oligonucleotides labelled BSA aqueous solution (solution 2) to the conjugation buffer in the second flask with stirring at room temperature;
14. Addition of 100 ml colloidal gold solution into each flask with stirring at room temperature;
15. Addition of 200 ml of stabilizing buffer to each flask and concentrate each conjugate by cooled (temperature around 15° C.). high speed centrifugation (10,000 rpm for one hour);
16. Discarding the supernatant and resuspending the concentrated conjugates at room temperature;
17. Adjusting the concentration for each of the two conjugates to O.D.₅₂₀=2.0;
18. Addition of 0.1 ml of Tween 20 only to the first conjugate and soaking glass fibre sheet conjugate pad into the conjugate, then heating dry at temperature around 50° C.
19. Soaking another glass fiber sheet conjugate pad into the second conjugate, then heating dry at temperature around 50° C.
20. Printing of sample (e.g. anti-αhCG) and control lines onto the nitrocellulose membranes.
21. Lamination of cards using the first gold conjugate. Lamination of card components onto the backing material with the sequence (in case of conjugate releasing site laminated within the upper side of the device plastic housing):
   a. Laminate the nitrocellulose membrane nearly in the middle of the card.
   b. Laminate the absorbent pad in the end of the card (overlaps from the nitrocellulose membrane side).
   c. Laminate the first conjugate pad in the other side of the nitrocellulose membrane.
   d. Laminate the sample pad.

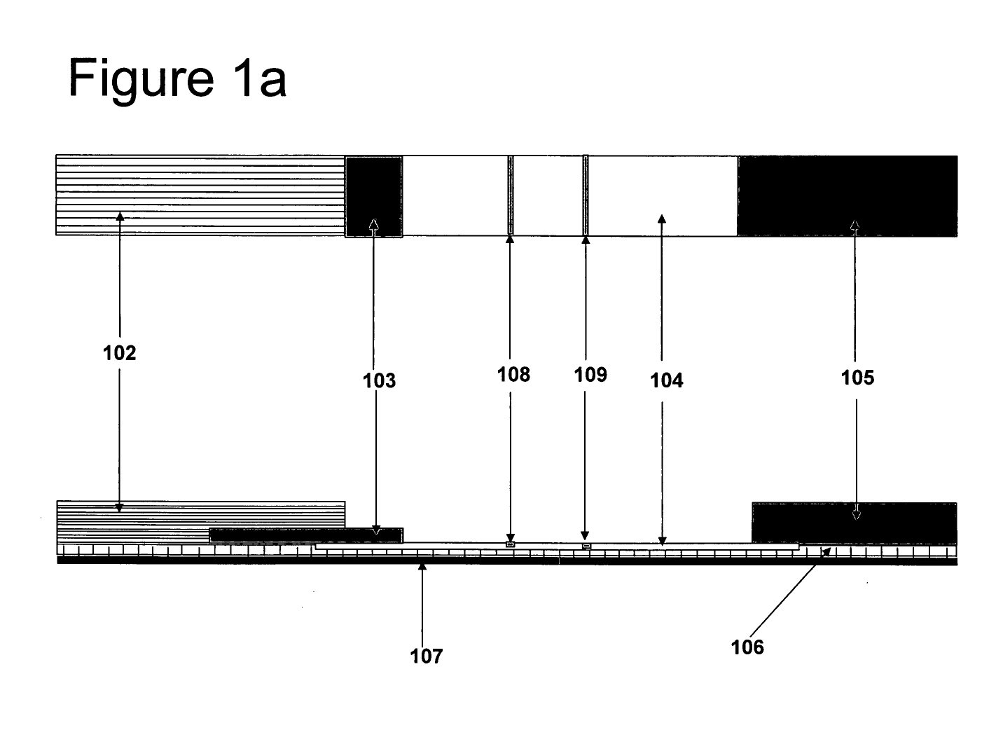

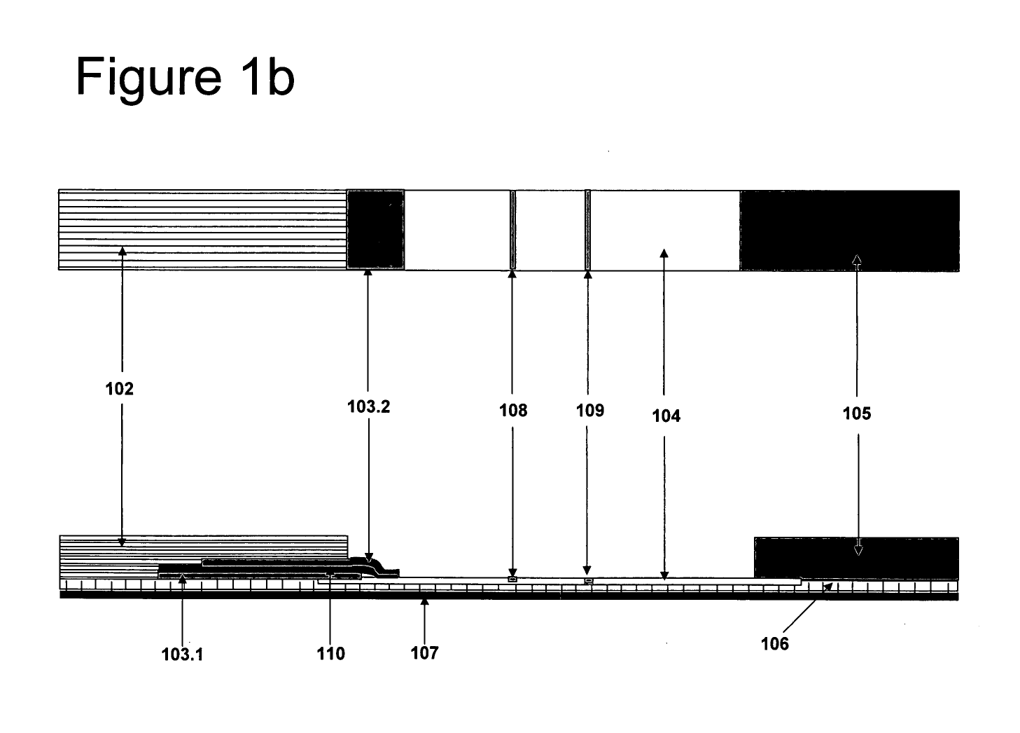

Lamination of card components onto the backing material with the sequence (in case of conjugate releasing site laminated onto the test strip itself separated from the first conjugate by a divider), see FIG. 1b:
a. Laminate the nitrocellulose membrane nearly in the middle of the card.
b. Laminate the absorbent pad in the end of the card (overlaps from the nitrocellulose membrane side).
c. Laminate the first conjugate pad in the other side of the nitrocellulose membrane.
d. Laminate the plastic divider onto the first conjugate (overlaps from the nitrocellulose membrane side).
e. Laminate the second conjugate pad onto the divider (overlaps from the nitrocellulose membrane side).
f. Laminate the sample pad onto the other end of the card, the sample pad will overlaps with the two conjugate pads.
22. Then cut cards into strips.
23. Lamination of the second gold conjugate will applied within the plastic housing itself to ensure that the two conjugates will not propagate before release from the releasing pad and so stick within the releasing pad.

The first conjugate releasing pad 103.1 is laminated on the test strip between the sample pad and the nitrocellulose membrane while the second 103.2 is above the first pad separated by a divider 110 to be released directly toward the nitrocellulose membrane without flow through the first conjugate pad to avoid interact with the first conjugate before reaching the membrane, see FIG. 1b. The second conjugate releasing site could be laminated within the upper side of the device plastic housing.
24. In case of a detection cup as the test device for urine or saliva, the two gold conjugates are fixed in different positions into the internal surface of the cup, for the same reason as in 13, see FIGS. 5 and 6.

Finally the lamination of the second gold conjugate will be applied within the plastic housing itself to ensure that the two conjugates will not propagate before release from the releasing pad and so stick within the releasing pad.

* In case of antibodies/antigens and their specific antigens/antibodies there is no need for these steps of bovine serum albumin or any other protein labeling.

** Other proteins or peptides could be used other than bovine serum albumin

### Example 3

### Pregnancy Detection System

The first gold conjugate is made of mouse anti-βhCG and four oligonucleotides conjugated with colloidal gold conjugate, and the second gold conjugate is the conjugate of the four complementary oligonucleotides. The first gold conjugate 103.1 was laminated in the side of the nitrocellulose membrane, while the second gold conjugate 103.2 is laminated above the first pad 103.1 separated by a divider 110 that enables the second conjugate to take a part of the sample and release directly onto the nitrocellulose membrane.

The plastic housing is the plastic design where we insert the test strip. The first conjugate releasing pad 103.1 is laminated on the test strip between the sample pad and the nitrocellulose membrane while the second 103.2 is above the first pad separated by a divider 110 to be released directly toward the nitrocellulose membrane without flow through the first conjugate pad to avoid interact with the first conjugate before reaching the membrane, see FIG. 1b. The second conjugate releasing site could be laminated within the upper side of the device plastic housing.

The sample line is a mouse anti-αhCG immobilized onto the nitrocellulose membrane. The control line is anti-mouse IgG. Sample and control lines turn into purple color in case of hCG availability in the sample; only the control line turns into purple color in case of hCG free sample.

The commercially available rapid tests sensitivity for the pregnancy hormone which is human chorionic gonadotropin hormone (hCG) is around 25 mIU/ml while according to this system it is so simple to detect less than 1 mIU/ml.

### Example 4

### Hepatitis B Surface Antigen (HBsAg) Detection System

The first gold conjugate is made of mouse anti-HBsAg (clone 1) and four oligonucleotides conjugated with colloidal gold conjugate, and the second gold conjugate is the conjugate of the four complementary oligonucleotides. The numbering of clones are only for explanation and to recognize that we use always two different clones of monoclonal antibodies; these two monoclonal antibodies capture the target antigen from two different sites, so we call them a pair of monoclonal antibodies. The first gold conjugate 103.1 was laminated in the side of nitrocellulose membrane 104, while the second gold conjugate 103.2 was laminated above the first pad 103.1 separated by a divider 110 that enables the second conjugate to take a part of the sample and release it directly onto the nitrocellulose membrane.

The plastic housing is the plastic design, where we insert the test strip. The first conjugate releasing pad 103.1 is laminated on the test strip between the sample pad and the nitrocellulose membrane while the second 103.2 is above the first pad separated by a divider 110 to be released directly toward the nitrocellulose membrane without flow through the first conjugate pad to avoid interact with the first conjugate before reaching the membrane, see FIG. 1b. The second conjugate releasing site could be laminated within the upper side of the device plastic housing.

The sample line 108 is mouse anti-HBsAg (clone 2) immobilized onto the nitrocellulose membrane 104. The control line 109 is anti-mouse IgG. Sample 108 and control lines 109 turn into purple color in case of HBsAg availability in the sample; only the control line 109 turns into purple color in case of HBsAg free sample, see FIG. 1b.

The commercially available rapid tests sensitivity for hepatitis B surface antigen is within the range 500-1000 pg/ml while according to this system it is so simple to detect less than 10 pg/ml.

### Example 5

### Human Immunodeficiency Virus (HIV) Antibodies Detection System

The first gold conjugate is mouse anti-human Immunoglobulin G (anti-hIgG) and four oligonucleotides conjugated with colloidal gold conjugate, and the second gold conjugate is the conjugate of the four complementary oligonucleotides. The first gold conjugate 103.1 was laminated in the side of nitrocellulose membrane 104, while the second gold conjugate 103.2 was laminated above the first pad 103.1 separated by a divider that enables the second conjugate to take a part of the sample and release it directly onto the nitrocellulose membrane 104.

The plastic housing is the plastic design where we insert the test strip. The first conjugate releasing pad 103.1 is laminated on the test strip between the sample pad and the nitrocellulose membrane while the second 103.2 is above the first pad separated by a divider 110 to be released directly toward the nitrocellulose membrane without flow through the first conjugate pad to avoid interact with the first conjugate before reaching the membrane, see FIG. 1b. The second conjugate releasing site could be laminated within the upper side of the device plastic housing.

The sample line 108 is a combination of synthetic or recombinant HIV antigen immobilized onto the nitrocellulose membrane 104. The control line 109 is anti-mouse IgG. Sample 108 and control 109 lines turn into purple color in case of HIV antibodies availability in the sample; only the control line 109 turns into purple color in case of HIV antibodies free sample, see FIG. 1b.

According to this system it is so simple to detect very low titers of HIV antibodies in serum.

The features disclosed in the foregoing description, in the claims and/or in the accompanying drawings may, both separately and in any combination thereof, be material for realizing the invention in diverse forms thereof.

### REFERENCES

(1) J Chandler, N Robinson, and K Whiting, “Handling False Signals in Gold-Based Rapid Tests”, IVD Technology 7, no. 2 (2001): 34-45; http://www.devicelink.com/ivdt/archive/01/03/002.html.
(2) J Chandler, T Gurmin, and N Robinson, “The Place of Gold in Rapid Tests”, IVD Technology 6, no. 2 (2000): 37-49; http://www.devicelink.com/ivdt/archive/00/03/004.html
(3) TC Tisone et al., “Image Analysis for Rapid-Flow Diagnostics”, IVD Technology 5, no. 5 (1999): 52-58; http://www.devicelink.com/ivdt/archive/99/09/010.html.
(4) World Health Organization, HIV assays: operational characteristics (Phase I). Report 13: urine specimens, oral fluid (saliva) specimens. [Material originally distributed as WHO/BCT/02.08]
(5) Zaaijer, H. L., Exel-Oehlers, P. V., Kraaijeveld, T., Altena, E., Lelie, P. N. (1992) Early detection of antibodies to HIV-1 by third-generation assays. Lancet 340, 770-772.
(6) Constantine, N. T., van der Groen, G., Belsey, E. M., Tamashiro, H. (1994) Sensitivity of HIV-antibody assays determined by seroconversion panels. AIDS 8, 1715-1720.
(7) Journal of Microbiological Methods, 45, 2001: 41-52.
(8) Duncan, R. J. S., Weston, P. D., Wrigglesworth, R., (1983) A new reagent which may be used to introduce sulfhydryl groups into proteins, and its use in the preparation of conjugates for immunoassay. Anal. Biochem. 132, 68.

1. A method for rapid immunochromatographic detection of a target in a sample comprising the step of forming a sandwich, by contacting
(i) a first colloidal gold conjugate conjugated with a specific antibody or antigen and at least one oligonucleotide or non-specific antibody or antigen, and
(ii) a second colloidal gold conjugate conjugated with at least one complementary oligonucleotide or non-specific antibody or antigen.

2. The method according to claim 1, comprising the following steps:
(a) applying the sample to a sample application site,
(b) allowing the target in the sample getting captured by the first colloidal gold conjugate from a target site at a first conjugate releasing site,
(c) allowing the target in the sample to move to a test zone getting captured by another immobilised specific antibody or antigen from another target site,
(d) allowing to release the second colloidal gold conjugate from a second conjugate releasing site for capturing the first colloidal gold conjugate from at least one oligonucleotide site or non-specific antibody or antigen site,
(e) allowing the sample to move through the test zone and a control zone to an absorbent site,
(f) allowing to continuously release the first and the second colloidal gold conjugates from the first and the second conjugate releasing sites to propagate to the test zone and the control zone,
(g) detecting presence or absence of a color in the control zone, and
(h) detecting presence or absence of a color in the test zone.

3. The method according to claim 1, wherein the specific antibody or antigen is selected from the group consisting of anti-beta chorionic gonadotropin hormone (anti-βhCG), anti-lipoarabinomannan (LAM), hepatitis virus antibodies against or antigens from hepatitis virus type A, hepatitis virus type B, or hepatitis virus type C or human immunoglobulin G antibodies or antigens.

4. The method according to claim 3, wherein the hepatitis virus antigen is hepatitis B surface antigen (HBsAg) and the hepatitis virus antibody is anti-HBsAg.

5. The method according to claim 1, wherein another specific antibody or antigen is selected from the group consisting of anti-alpha chorionic gonadotropin hormone (anti-αhCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-0 or HIV-1-M.

6. The method according to claim 5, wherein the hepatitis virus antigen is hepatitis B surface antigen (HBsAg), the hepatitis virus antibody is anti-HBsAg and the human immunodeficiency virus (HIV) antigen is HW p160.

7. The method according to claim 1, wherein the sample comprises a body fluid of a subject.

8. The method according to claim 7, wherein the body fluid is selected from the group consisting of urine, whole blood, serum, plasma and saliva.

9. A test device for conducting the method for rapid immunochromatographic detection of a target in a sample of claim 1, the test device comprising a housing comprising a test strip (101) comprising a sample application site (102); a first conjugate releasing site (103.1); a second conjugate releasing site (103.2); a nitrocellulose membrane (104); a test zone (108) and a control zone (109); and a sample absorbent site (105).

10. The test device according to claim 9, wherein the test strip (101) is attached to a supporting backing (107) by means of an adhesive (106).

11. The test device according to claim 10, wherein the supporting backing (107) is a plastic backing.

12. The test device according to claim 9, wherein the test zone (108) comprises another specific antibody or antigen.

13. The test device according to claim 12, wherein another specific antibody or antigen is selected from the group consisting of anti-alpha chorionic gonadotropin hormone (anti-αhCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-O or HIV-1-M.

14. The test device according to claim 13, wherein the hepatitis virus antigen is hepatitis B surface antigen (HBsAg), the hepatitis virus antibody is anti-HBsAg and the human immunodeficiency virus (HIV) antigen is HIV p160.

15. The test device according to claim 9, wherein the second conjugate releasing site (103.2) is laminated within the upper side of the housing.

16. A test device for conducting the method for rapid immunochromatographic detection of a target in a sample of claim 1, the test device comprising a detection cup (510).

17. The test device according to claim 16, wherein the detection cup (510) comprises:
(a) a sample collecting container (501);
(b) a test strip comprising a sample application site (404); a nitrocellulose membrane (401); two test zones (403) and a control zone (402); and a sample absorbent site (405); and
(c) a first conjugate releasing site (503) and a second conjugate releasing site (504) separated from the test strip.

18. The test device according to claim 17, wherein the test zones (403) comprise another specific antibody or antigen.

19. The test device according to claim 18, wherein another specific antibody or antigen is selected from the group consisting of anti-alpha chorionic gonadotropin hormone (anti-ahCG), anti-lipoarabinomannan (LAM), hepatitis virus antibodies against or antigens from hepatitis virus type A, hepatitis virus type B, or hepatitis virus type C or human immunodeficiency virus (HIV) antibodies or antigens from the HIV type HIV-1 and HIV-2 or HIV subtype HIV-1-N, HIV-1-O or HIV-1-M.

20. The test device according to claim 19, wherein the hepatitis virus antigen is hepatitis B surface antigen (HBsAg), the hepatitis virus antibody is anti-HBsAg and the human immunodeficiency virus (HIV) antigen is HIV p160.

21. The method according to claim 1, wherein said method comprises for diagnosing and monitoring a disease or a specific condition of a subject by detecting a target indicative of the disease or specific condition in a sample from the subject.

22. The method according to claim 21, wherein the specific condition is pregnancy.

23. The method according to claim 21, wherein the target is human chorionic gonadotropin hormone (hCG).

24. The method according to claim 21, wherein the disease is hepatitis selected of the group consisting of hepatitis type A, hepatitis type B, or hepatitis type C.

25. The method according to claim 24, wherein the selected hepatitis type is hepatitis type B.

26. The method according to claim 21, wherein the target is hepatitis B surface antigen (HBsAg).

27. The method according to claim 21, wherein the disease is an HIV infection selected from the HIV infection group consisting of HIV type HIV-1 and HIV-2 or selected from the group consisting of HIV subtype HIV-1-N, HIV-1-O and HIV-1-M.

28. The method according to claim 21, wherein the target is selected from an HIV antibody or antigen selected from the group consisting of p41, p120, p160, p18, p24/25, p55, p34, p40, p52, p68.

29. The method according to claim 28, wherein the HIV antigen is p160.

30. A kit for rapid immunochromatographic detection of a target in a sample comprising the test device according to any of claims 9 to 15 or the test device according to any of claims 16 to 20 and at least one of further reagents, was buffers and instructions for use.

31. (canceled)

32. (canceled)

33. A method for the manufacture of the test device according to claim 9, comprising the following steps:
(a) preparing a first colloidal gold conjugate by adding a specific antibody or antigen to a conjugation buffer with at least one oligonucleotide or non-specific antibody or antigen and then adding it to a colloidal gold solution,
(b) preparing a second colloidal gold conjugate by adding at least one complementary oligonucleotide or non-specific antibody or antigen in a conjugation buffer to a colloidal gold solution, and
(c) preparing a first conjugate releasing site and a second conjugate releasing site by applying the first and the second gold conjugate on different pads.

34. A method for the manufacture of the test device according to claim 9, comprising the following steps: of
(a) preparing a sample application site, a test zone, a control zone and a sample absorbent site,
(b) assembling the sample application site, the test zone, the control zone, the sample absorbent site together with the first and the second conjugate releasing sites on a test strip,
(c) applying the first and the second gold conjugates on different sites of the same card separated by a divider, and
(d) assembling the test strip in a housing.

35. A method for the manufacture of the test device according to claim 9, further comprising the following steps:
(a) preparing a sample application site, a test zone, a control zone and a sample absorbent site,
(b) assembling the sample application site, the test zone, the control zone, the sample absorbent site together with the first conjugate releasing site on a test strip, and
(c) assembling the test strip and the second conjugate releasing site in a housing.

36. A method for the manufacture of the test device according to claim 9, comprising the following steps:
(a) preparing a sample application site, two test zones, a control zone and a sample absorbent site,
(b) assembling the sample application site, the test zones, the control zone, and the sample absorbent site on a test strip, and
(c) assembling the test strip and the first and second conjugate releasing sites separately in a detection cup.
